(19) United States
(12) Patent Application Publication
(10) Pub. No.: US 2018/0280285 A1
(43) Pub. Date: Oct. 4, 2018

(54) HAIR PRODUCT KIT AND METHOD OF USE

(71) Applicant: Tsehai Lewis, Hartsdale, NY (US)

(72) Inventor: Tsehai Lewis, Hartsdale, NY (US)

(21) Appl. No.: 15/881,746

(22) Filed: Jan. 27, 2018

Related U.S. Application Data
(60) Provisional application No. 62/478,209, filed on Mar. 29, 2017.

(51) Int. Cl. A61K 8/92; A61Q 5/12

(52) U.S. Cl. CPC A61K 8/922; A61K 2800/5922; A61Q 5/12

(57) ABSTRACT

A method to treat hair includes in a functional order: wetting hair; applying a shampoo liquid to hair; rinsing hair; drying hair; applying henna liquid to hair; covering hair with a shower cap; applying heat to hair; removing the shower cap; rinsing henna liquid from hair; applying deep conditioner to hair; covering hair with the shower cap; applying heat to hair; removing the shower cap; rinsing deep conditioner; drying hair; and applying a small amount of leave in conditioner to hair

### BACKGROUND

### 1. Field of the Invention

The present invention relates generally to hair products and methods of manufacturing the same.

### 2. Description of Related Art

Liquid hair products are well known in the art and provide means for a cleaning a user's hair. Great strides in the area of hair products have been made; however, many shortcomings remain and opportunities to develop better hair products exist. Accordingly, the present invention is an improvement on existing liquid hair products.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIGS. 1-3 are flowcharts depicting the preferred method of use in accordance with the preferred ingredients for the liquid hair product.

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

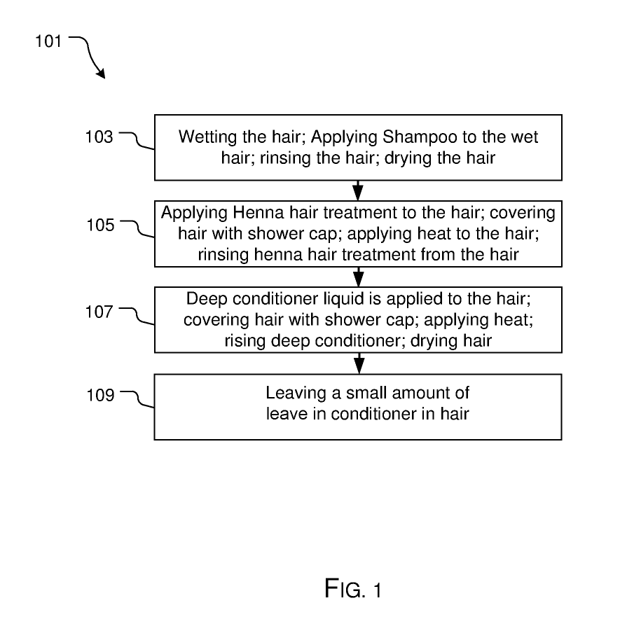

Referring now to the drawings wherein like reference characters identify corresponding or similar elements throughout the several views, FIGS. 1-3 depict flowcharts of the preferred method of use along with a kit and hair treatment solution in accordance with a preferred embodiment of the present invention.

The present invention is related to a hair product kit in which the components work in conjunction with one another to produce an efficient and simple way to effectively produce a consistent henna hair treatment each time its used according to sequence listed in the directions, as found flowchart 101 of FIG. 1.

As shown in box 103, the user's hair is made wet and shampoo is applied thereto. The process includes lathering the hair with the shampoo liquid. Next, the hair is rinsed and the process is repeated.

As shown in box 105, henna liquid is applied to the towel-dried shampooed hair. It can be applied using gloved hands, which are included in the kit. A shower cap can be used to put over the hair after the henna liquid has been thoroughly applied to hair. Next, the user sits under an overhead dryer with henna in hair, covered by a plastic cap for 30 minutes.

After 30 minutes, the henna liquid is rinsed out of hair, until all visible traces are removed. As shown in box 107, the hair is towel dried and the deep conditioner liquid is applied to the hair. The user then applies a second shower cap after deep conditioner is applied to hair. The user sits under an overhead dryer for 15 minutes. After the 15 minutes, the deep conditioner is rinsed out, and the hair is towel dried. After this process, the last step is to apply a small amount of leave in conditioner, it is not rinsed out, as shown in box 109. Using the steps in the sequence above ensure proper application and desirable results.

It should be understood that the system includes four components: (1) Shampoo liquid, (2) Henna liquid, (3) Deep Conditioner liquid, and (4) the process of leaving in the treatment.

It should be understood that the Henna liquid is a natural treatment for hair to make it thicker, stronger and to dye hair naturally. It can be difficult to get desirable results because of the nature of the treatment and what it can do to hair if not used properly. Henna when applied to hair behaves like a protein treatment. Proteins on hair are great for strength but can cause excessive drying of hair. The present system alleviates that dryness by adding moisture before and after the henna treatment to ensure maximum moisture is retained.

Other advantages of the present system is its efficiency. The system provides a ready to use, easy to apply henna formula that requires no time for the dye to release.

In the present system, the process includes utilizing body quality art henna, which has the most lawsone content. It should be understood that lawsone is the naturally occurring dye found in henna leaves. The type of henna used in this system produces, deep penetrating color, that penetrates to the hair shaft and binds with the keratin in the hair.

It should be understood that the recommended time to allow for dye release with traditional henna mixes is 3 hours. After the 3 hours, the henna mix has to be applied for 2 hours with traditional henna mixes to allow for adequate penetration to hair. The present system has minimized the 5-hour process to a mere 30 minutes with heat, under an overhead dryer with a shower cap. A special oil complex has been added at the right stages of the henna process to ensure proper hair care. If the oils are added at the wrong stage in the henna process, the henna molecules are unable to penetrate the hair shaft, thereby nullifying the effect of the henna.

The present invention is a moisturizing, sulfate free shampoo that will properly prepare the hair for the henna. It cleans the hair without stripping and adds moisture so that the henna does not dry out the hair while its applied to the hair. A special oil blend has been added to the shampoo to aid in more moisture and proper care of hair before the henna is applied. A special botanical complex has also been added to the shampoo that aids in hair growth and darkening gray hairs. After the hair is shampooed, with this special, protein free formula it is ready for the henna to be applied.

The present invention includes a henna formula that is ready to use. It comes in a squeezable tube, which decreases the mess of traditional mixes. This henna formula is made with only Body Quality Art henna powder, which is the highest quality henna you can use. It is PPD free and does not have the allergens found in some cheaper, commercially distributed henna dyes on the market now. No oils are added to this henna formula, as the oils act as a barrier and do not allow adequate penetration of henna into the hair shaft. A botanical complex rich in antioxidants has been added, which promotes hair growth, and aids in better gray hair coverage. The consistency is smooth and applies to hair in a uniform way, unlike some traditional mixes. It does not drip like traditional henna mixes and makes for less mess. This henna formula stays where it is applied.

The present invention includes a protein free, deep conditioner to add softness, moisture and ease of combing after henna treatment is applied. Hair can be hard and tangled after henna is applied, with traditional henna mixes. The system adds a special oil complex to return moisture back to hair after the henna has been washed out. A special botanical complex, rich in antioxidants has been added, which promotes hair growth, and aids in better gray coverage. Using the wrong conditioner after you henna can cause unwanted hair breakage due to dry, brittle hair. This deep conditioner alleviates this problem, giving the hair what it needs after a henna treatment.

The present invention includes a moisturizing leave in conditioner to use after the deep conditioner. This will add moisture to hair. It is not rinsed out, and helps add and retain moisture in hair. It has conditioners to add ease of combing while hair is wet. This leave in conditioner contains my special oil complex to further treat hair after the deep conditioner has been rinsed out. This leave in conditioner also contains a special botanical complex, rich in antioxidants which aid in hair growth and aid in dyeing gray hair.

The present invention tackles all the problems that can occur with the application of traditional henna mixes from start to finish. The present invention takes the guess work out when it comes to henna and created a convenient kit, so that doing a henna hair treatment can be efficient and uncomplicated.

It should be understood that the leave in conditioner is used in a small amount, and in one embodiment, the size of a quarter. The process is necessary because it is left in the hair aiding in replenishing the moisturizers that are washed out when the deep conditioner is washed out.

In the preferred embodiment it is required to utilize a sulfate free shampoo with oils, and the deep conditioner needs to be protein free as a deep conditioner with protein would not work after the henna process.

In one embodiment, green tea is added for its antioxidant purposes, for marketing claims, and health claims that it does indeed aid in hair growth. In one embodiment, sage could also be utilized because of it is a natural extract to help in dying grays.

It should be understood that the henna is intended to dye gray hairs, too. It should be understood that home made henna does drip, which in turn causes a mess. In the present invention, the henna formula does not drip and was made that way on purpose.

Accordingly, the shampoo must be sulfate free for optimal hair prior to henna application. The henna must be oil free. The formulation is such that it is moist enough to penetrate the hair, but not so much so that it drips from the hair and scalp. The deep conditioner must be protein free. And, the leave in conditioner must be used so that whatever moisturizers that were washed out when the deep conditioner was rinsed out are replaced but with a formulation that does not weight the hair down, but still provided optimal moisture, easy of combing and softness.

The system is developed to be convenient. Each kit will come with 2 shower caps and 2 pairs gloves so that a person has all they need to do a henna treatment at home. The kit is intended for home use. An overhead dryer is essential to allow the henna to effectively and efficiently penetrate the hair shaft.

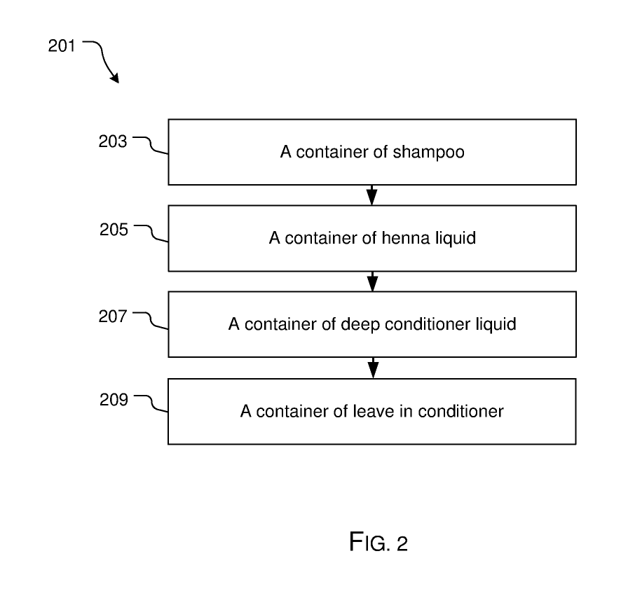

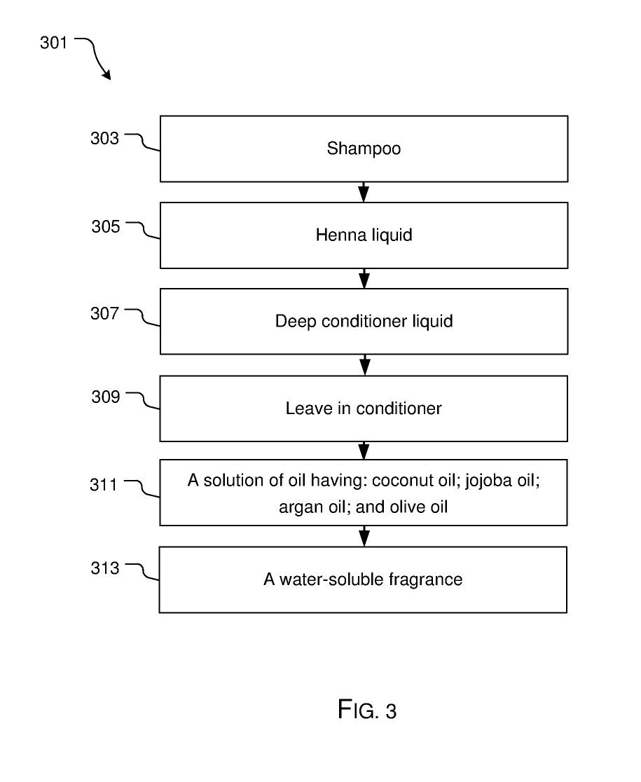

In the preferred embodiment, the oils added to the shampoo, deep conditioner and leave in conditioner are: (1) coconut oil, which has high moisture retaining capacity does not easily evaporate which keeps hair soft and moist, adds shine smoothness and strengthens hair; (2) Jojoba oil, which moisturizes hair without residue, readily accepted by scalp as it hair similar molecular structure to sebum the scalp's natural oil and increases hair elasticity and helps repair damaged hair; (3) Argan oil, which softens hair, helps treat split ends and tame frizzy hair, makes hair more manageable and adds sheen; and (4) Olive oil which moisturizing helps alleviate dry hair, softens hair, reduces frizz, makes hair stronger and shinier. The hair treatment kit and the solution are found in FIGS. 2 and 3 and depicted in flowcharts 201, 301 and respective boxes 203-209 and 303-313.

It should be understood that many people believe henna to have an unpleasant odor. A pleasant smelling water-soluble fragrance was added the henna mix that does not interfere with its functionality, as traditional oil-based fragrances would.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. A method to treat hair, comprising in a functional order:
wetting hair;
applying a shampoo liquid to hair;
rinsing hair;
drying hair;
applying henna liquid to hair;
covering hair with a shower cap;
applying heat to hair;
removing the shower cap;
rinsing henna liquid from hair;
applying deep conditioner to hair;
covering hair with the shower cap;
applying heat to hair;
removing the shower cap;
rinsing deep conditioner;
drying hair; and
applying a small amount of leave in conditioner in hair.

2. A hair treatment solution, comprising:
shampoo;
henna liquid;
deep conditioner liquid;
leave in conditioner;
a solution of oil, having:
coconut oil;
jojoba oil;
argan oil;
olive oil; and
a water-soluble fragrance.

3. The solution of claim 2, wherein the shampoo is sulfate free.

4. The solution of claim 2, wherein the henna is oil free.

5. The solution of claim 2, wherein the solution is moist enough to penetrate hair and drip-free.

6. The solution of claim 2, wherein the deep conditioner is protein free.

7. A hair treatment kit, comprising:
a container of shampoo;
a container of henna liquid;
a container of deep conditioner liquid; and
a container of leave in conditioner.

8. The hair treatment kit of claim 7, further comprising:
a shower cap.

9. The hair treatment kit of claim 7, wherein the shampoo is sulfate free.

10. The hair treatment kit of claim 7, wherein the henna is oil free.

11. The hair treatment kit of claim 7, wherein the solution is moist enough to penetrate hair and drip-free.

12. The hair treatment kit of claim 7, wherein the deep conditioner is protein free.
